(19) United States
(12) Patent Application Publication
(10) Pub. No.: US 2026/0182926 A1
(43) Pub. Date: Jul. 2, 2026

(54) SYSTEM AND METHOD FOR ACQUIRING HIGH-RESOLUTION AMPLIFIED BIOLOGICAL ACTIVITY SIGNAL

(71) Applicant: JCF TECHNOLOGY INC., Seoul (KR)

(72) Inventor: Jin Myung KIM, Bucheon-si, Gyeonggi-do (KR)

(21) Appl. No.: 18/840,910

(22) PCT Filed: Apr. 10, 2024
(86) PCT No.: PCT/KR2024/004782

§ 371 Date: Aug. 23, 2024

(30) Foreign Application Priority Data
Apr. 11, 2023 (KR) 10-2023-0047854

(51) Int. Cl. A61B 5/00; A61B 5/024; A61B 5/05; A61B 5/08

(52) U.S. Cl. CPC A61B 5/7228; A61B 5/024; A61B 5/05; A61B 5/0816; A61B 5/7225

(57) ABSTRACT

Provided are a system and a method for acquiring a high-resolution amplified biological activity signal. The system for acquiring a high-resolution amplified biological activity signal according to an embodiment of the present invention includes: a biological activity signal processing unit for generating a preprocessed biological activity signal by acquiring a biological activity signal of a user using a radar device and performing preprocessing on the biological activity signal; a digital signal processing unit for generating a digital-processed biological activity signal by performing digital-processing on the preprocessed biological activity signal; and a signal extraction unit for outputting the digital-processed biological activity signal to extract an amplified signal for each preset biological activity signal category.

### TECHNICAL FIELD

The present invention relates to a system and method for acquiring a high-resolution amplified biological activity signal, and particularly, to a system and method for acquiring a high-resolution amplified biological activity signal, which is formed to output information on biological activities of a user acquired through radars, after amplifying the biological activity information to have a resolution higher than an interpretable level by controlling an amplification degree according to acquired biological activity signals.

### BACKGROUND ART

Recently, the number of people in need of care or attention is increasing in the domestic society. The people in need of care or attention may be defined as people who need assistance when an emergency situation occurs or people who do not have a housemate, and for example, the elderly, the disabled, and single-person households may be included. According to a recent survey, the elderly population has exceeded 9 million, and the population of single-person households and registered disabled people has also exceeded 6.6 million and 2.6 million, respectively. In addition, the number of people who die alone due to absence of a housemate exceeds 10,000.

In addition, together with increase in the number of personal terminals and demands for personal health management, various solutions or applications that collect personal healthcare data and provide personalized health management contents using the personal healthcare data are emerging one after another recently. Wearable devices or installation-type devices are used to collect such individual healthcare data. When the wearable devices are used, preprocessing of measured data is not needed in many cases since healthcare data is acquired from the parts close to the body. However, in the case of the installation-type devices, since physical activity information of a user is measured from a distance, there is a problem in that the probability of mixing measured data with noise increases, and it is difficult to distinguish a subtle difference.

### DISCLOSURE OF INVENTION

### Technical Problem

In order to solve the problems of the prior art as described above, an embodiment of the present invention provide a system and method for acquiring a high-resolution amplified biological activity signal, which can variably determine magnitude of amplification with respect to biological activity signals that change in real time by determining the magnitude of amplification using a preset noise ratio and an amplification degree and amplifying the signals, even when low-frequency and low-resolution biological activity signals are acquired through radars.

### Technical Solution

To accomplish the above object, according to one aspect of the present invention, there is provided a high-resolution amplified biological activity signal acquisition system. The high-resolution amplified biological activity signal acquisition system comprises: a biological activity signal processing unit for generating a preprocessed biological activity signal by acquiring a biological activity signal of a user using a radar device and performing preprocessing on the biological activity signal; a digital signal processing unit for generating a digital-processed biological activity signal by performing digital-processing on the preprocessed biological activity signal; and a signal extraction unit for outputting the digital-processed biological activity signal to extract an amplified signal for each preset biological activity signal category.

The biological activity signal processing unit may include: a biological activity signal acquisition module for acquiring the biological activity signal including a first biological activity signal that is a biological signal and a second biological activity signal that is an activity signal; and a biological activity signal preprocessing module for generating the preprocessed biological activity signal by performing preprocessing on the biological activity signal.

The preprocessing may be a process of performing amplification at a preset magnitude using an amplifier on each of the first biological activity signal and the second biological activity signal, and applying a low pass filter of a preset band.

The digital signal processing unit may include: a signal conversion module for digitalizing the preprocessed biological activity signal; a signal ratio calculation module for confirming an amplification degree of a digital biological activity signal, which is the digitalized preprocessed biological activity signal, and calculating a signal-to-noise ratio by performing conversion on the digital biological activity signal; and an amplification magnitude determination module for determining the preset magnitude by applying the amplification degree and the signal-to-noise ratio to a preset algorithm.

The signal extraction unit may include: a digital-processed biological activity signal output determination module for determining to output the digital biological activity signal when the signal-to-noise ratio is higher than a preset magnitude, and not to output the digital biological activity signal when the signal-to-noise ratio is lower than the preset magnitude; and a digital-processed biological activity signal output module for determining, when it is determined to output the digital-processed biological activity signal, the preset biological activity signal category to which the digital-processed biological activity signal corresponds, and separately outputting the digital-processed biological activity signal in each signal category.

According to another aspect of the present invention, there is provided a high-resolution amplified biological activity signal acquisition method. The high-resolution amplified biological activity signal acquisition method comprises: a biological activity signal processing step of generating a preprocessed biological activity signal by acquiring a biological activity signal of a user using a radar device and performing preprocessing on the biological activity signal, by a biological activity signal processing unit; a digital signal processing step of generating a digital-processed biological activity signal by performing digital-processing on the preprocessed biological activity signal, by a digital signal processing unit; and a signal extraction step of outputting the digital-processed biological activity signal to extract an amplified signal for each preset biological activity signal category, by a signal extraction unit.

The biological activity signal processing step may include: a step of acquiring the biological activity signal including a first biological activity signal that is a biological signal and a second biological activity signal that is an activity signal; and a biological activity signal preprocessing step of generating the preprocessed biological activity signal by performing preprocessing on the biological activity signal.

The preprocessing may be a process of performing amplification at a preset magnitude using an amplifier on each of the first biological activity signal and the second biological activity signal, and applying a low pass filter of a preset band.

The digital signal processing step may include: a step of digitalizing the preprocessed biological activity signal; a signal ratio calculation step of confirming an amplification degree of a digital biological activity signal, which is the digitalized preprocessed biological activity signal, and calculating a signal-to-noise ratio by performing conversion on the digital biological activity signal; and an amplification magnitude determination step of determining the preset magnitude by applying the amplification degree and the signal-to-noise ratio to a preset algorithm.

The signal extraction step may include: a digital-processed biological activity signal output determination step of determining to output the digital biological activity signal when the signal-to-noise ratio is higher than a preset magnitude, and not to output the digital biological activity signal when the signal-to-noise ratio is lower than the preset magnitude; and a digital-processed biological activity signal output step of determining, when it is determined to output the digital-processed biological activity signal, the preset biological activity signal category to which the digital-processed biological activity signal corresponds, and separately outputting the digital-processed biological activity signal in each signal category.

### Advantageous Effects

The system and method for acquiring a high-resolution amplified biological activity signal according to an embodiment of the present invention has an effect of variably determining magnitude of amplification with respect to biological activity signals that change in real time by determining the magnitude of amplification using a preset noise ratio and an amplification degree and amplifying the signals, even when low-frequency and low-resolution biological activity signals are acquired through radars.

### BRIEF DESCRIPTION OF THE DRAWINGS

FIG. 1 is a block diagram showing a high-resolution amplified biological activity signal acquisition system according to an embodiment of the present invention.

FIG. 2 is a block diagram showing the biological activity signal processing unit of FIG. 1.

FIG. 3 is a block diagram showing the digital signal processing unit of FIG. 1.

FIG. 4 is a block diagram showing the signal extraction unit of FIG. 1.

FIG. 5 is a flowchart illustrating a high-resolution amplified biological activity signal acquisition method according to an embodiment of the present invention.

FIG. 6 is a flowchart illustrating step S11 of FIG. 5.

FIG. 7 is a flowchart illustrating step S13 of FIG. 5.

FIG. 8 is a flowchart illustrating step S15 of FIG. 5.

FIG. 9 is a conceptual view showing the present invention of FIGS. 1 and 5.

FIGS. 10A-10D are exemplary diagrams showing a method of setting of an amplification magnitude in an amplification magnitude determination module or an amplification magnitude determination step of the present invention.

### BEST MODE FOR CARRYING OUT THE INVENTION

Hereinafter, some embodiments of the present disclosure will be described in detail with reference to illustrative drawings. In adding reference numerals to components in each drawing, like components may have like reference numerals as much as possible although they are shown in different drawings. In addition, in describing the present embodiments, when it is determined that a detailed description of a related known configuration or function may obscure the gist of the present technical spirit, the detailed description may be omitted. When “comprise”, “have”, “configured of”, and the like mentioned in the specification are used, other parts may be added as long as “only” is not used. When a component is expressed in a singular form, it may also include the plural, unless specifically stated otherwise.

In addition, in describing the components of the present disclosure, terms such as first, second, A, B, (a), and (b) may be used. These terms are used only to distinguish the components from other components, and the nature, sequence, order, or number of the components are not limited by the terms.

When two or more components are described as being “connected”, “coupled”, or “combined” in describing the positional relationship of components, although two or more components may be directly “connected”, “coupled”, or “combined”, it should be understood that the two or more components are “connected”, “coupled”, or “combined” as other components are further “interposed”. Here, other components may be included in one or more of two or more components “connected”, “coupled”, or “combined” with each other.

In describing the relationship of temporal flows related to the components, operation methods, manufacturing methods, and the like, for example, when a temporal precedence relationship or a flow precedence relationship is described as “after”, “in succession to”, “next”, “before”, or the like, non-successive cases may also be included unless “immediately” or “directly” is used.

Meanwhile, when a numerical value about a component or information corresponding thereto (e.g., levels, etc.) is mentioned, although there is no explicit description separately, the numerical value or information corresponding thereto may be interpreted as including a range of error that may occur due to various factors (e.g., processing factors, internal or external shocks, noise, and the like).

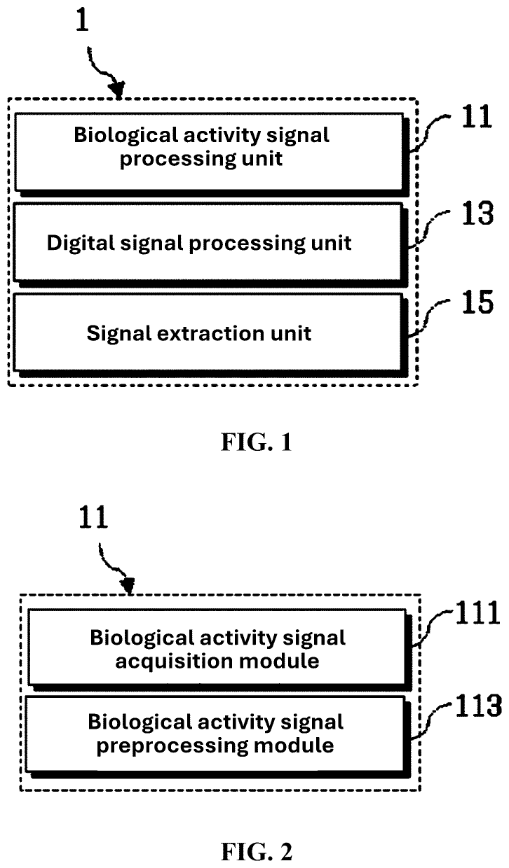

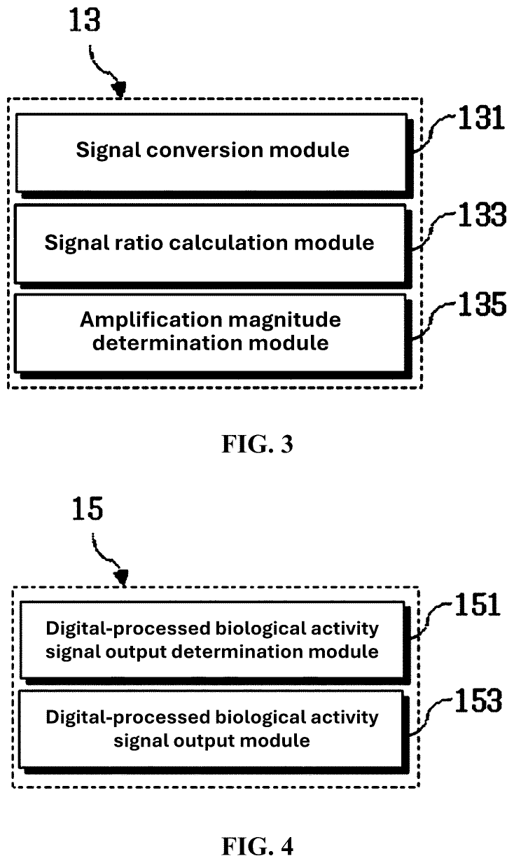

FIG. 1 is a block diagram showing a high-resolution amplified biological activity signal acquisition system according to an embodiment of the present invention, FIG. 2 is a block diagram showing the biological activity signal processing unit of FIG. 1, FIG. 3 is a block diagram showing the digital signal processing unit of FIG. 1, and FIG. 4 is a block diagram showing the signal extraction unit of FIG. 1. Hereinafter, a high-resolution amplified biological activity signal acquisition system according to an embodiment of the present invention will be described in detail with reference to FIGS. 1 to 4.

A high-resolution amplified biological activity signal acquisition system (1) according to an embodiment of the present invention may be formed to acquire biological activity signals of a user using a radar device, classify the acquired biological activity signals into preset categories, and amplify and output the signals in each category. To this end, the high-resolution amplified biological activity signal acquisition system 1 according to an embodiment of the present invention is formed to include a biological activity signal processing unit (11), a digital signal processing unit (13), and a signal extraction unit (15), as shown in FIG. 1.

The biological activity signal processing unit (11) according to an embodiment of the present invention is formed to generate a preprocessed biological signal by acquiring a biological activity signal of a user using a radar device and performing preprocessing on the biological activity signal. To this end, the biological activity signal processing unit (11) may be formed to include a biological activity signal acquisition module (111) and a biological activity signal preprocessing module (113), as shown in FIG. 2.

The biological activity signal acquisition module (111) is formed to acquire a biological activity signal of a user, which is information measured by a radar. The biological activity signal is formed to include a biological signal and an activity signal of the user. Here, the biological signal may be a signal measured for breathing, heart rate, and the like, and the activity signal may be a signal measured for movement of the user. When a biological activity signal is acquired, the biological activity signal acquisition module (111) may acquire the biological signal included in the biological activity signal as a first biological activity signal, and acquire the activity signal included in the biological activity signal as a second biological activity signal.

The biological activity signal preprocessing module (113) is formed to generate a preprocessed biological activity signal by performing preprocessing on the biological activity signal. Here, the preprocessing may be performed on each of the first biological activity signal and the second biological activity signal. The preprocessing performed by the biological activity signal preprocessing module (113) may be a process of performing amplification on each of the first biological activity signal and the second biological activity signal using an amplifier at a preset magnitude, and applying a low pass filter of a preset band. Here, although the preset magnitude may be an amplification magnitude determined by the digital signal processing unit (13), which will be described below, an initially determined amplification magnitude may be used as an exception for the first biological activity signal and the second biological activity signal acquired initially.

The biological activity signal preprocessing module (113) in another embodiment of the present invention may use a variable amplifier to amplify the first biological activity signal and the second biological activity signal. The variable amplifier of the present invention may further include a band pass function that acquires the first biological activity signal and the second biological activity signal and passes only the frequencies of a preset band, and perform a function of amplifying the frequency of a corresponding band at a preset magnitude. In addition, in this embodiment, the low-pass filter of a preset band described above may be formed, when the biological activity signal passing through the variable amplifier is included in a frequency band other than the frequency band intended to be used in the present invention, to exclude the frequency band.

However, the present invention is not necessarily limited thereto, and when the magnitude is preset in the biological activity signal preprocessing module (113), the determined amplification magnitude may be used by the digital signal processing unit (13) for initial amplification.

The digital signal processing unit (13) is formed to generate a digital-processed biological signal by performing digital-processing on the preprocessed biological activity signal. To this end, the digital signal processing unit (13) is formed to include a signal conversion module (131), a signal ratio calculation module (133), and an amplification magnitude determination module (135), as shown in FIG. 3.

The signal conversion module (131) is formed to digitalize the preprocessed biological activity signal. Here, the signal conversion module (131) may be, for example, an analog-to-digital converter (ADC). In addition, the signal conversion module (131) may be formed to further include a band pass filter to use a digital biological activity signal, which is the digitalized preprocessed biological activity signal converted through the analog-to-digital converter, only for a specific frequency band. Here, the specific frequency band of the band pass filter used by the signal conversion module (131) may be determined as a different frequency band according to the first biological activity signal and the second biological activity signal.

The digital biological activity signal, which is the preprocessed biological activity signal digitalized by passing through the signal conversion module (131), is used by the signal ratio calculation module (133) to confirm an amplification degree and calculate a signal-to-noise ratio. The signal ratio calculation module (133) may be formed to confirm the amplification degree of the digital biological activity signal and calculate the signal-to-noise ratio by performing conversion on the digital biological activity signal.

To this end, when a digital biological activity signal is acquired from the signal conversion module (131), first, the signal ratio calculation module (133) may acquire information on the amplification degree by confirming the amplification degree of the digital biological activity signal, and calculate a signal-to-noise ratio by performing conversion on the digital biological activity signal. The conversion used herein may be, for example, fast Fourier transform (FFT). The digital biological activity signal, to which the fast Fourier transform (FFT) is applied, is converted into a pure biological activity signal, and the signal ratio calculation module (113) may calculate the signal-to-noise ratio by comparing the digital biological activity signal and the pure biological activity signal.

The signal-to-noise ratio may be used by a signal output unit (15), which will be described below, to determine whether or not to output a corresponding digital biological activity signal according to the ratio value, and will be described below in further detail.

Meanwhile, the amplification magnitude determination module (135) may be formed to determine, when the signal-to-noise ratio is acquired, an amplification magnitude by applying the amplification degree and the signal-to-noise ratio to a preset algorithm. Here, the amplification magnitude may be an amplification magnitude set by the biological activity signal preprocessing module (113) as described above. The amplification magnitude determination module (135) may output the amplification magnitude as a PWM signal, and may be formed to control the amplification magnitude by outputting a voltage that may control the amplification degree through an integrator filter and providing the voltage to the amplifier of the biological activity signal preprocessing module (113).

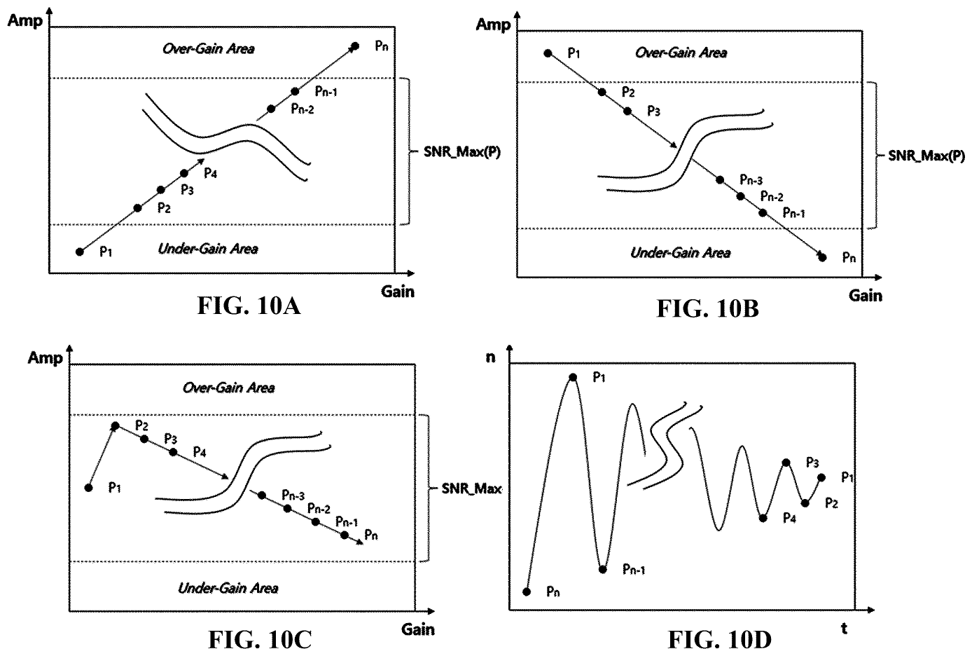

FIGS. 10A-10D are exemplary diagrams showing a method of setting of an amplification magnitude in an amplification magnitude determination module or an amplification magnitude determination step of the present invention. Referring to FIGS. 10A-10D, the amplification magnitude determination module (135) according to an embodiment of the present invention is configured to operate differently according to (a) a case of under-amplification, (b) a case of over-amplification (c) a case where optimization is required, and (d) a case where rapid setting to an SNR level is required.

First, in the case of under-amplification as shown in FIG. 10A, the amplification magnitude determination module (135) of the present invention may acquire an SNR (signal-to-noise ratio) by starting scanning from P1 and increasing the amplification degree to Pn, and determine an amplification magnitude by determining the maximum SNR point as the optimal amplification degree.

In addition, in the case of over-amplification as shown in FIG. 10B, contrary to FIG. 10A, the amplification magnitude determination module (135) may acquire an SNR by starting scanning from P1 and decreasing the amplification degree to Pn, and set the maximum SNR point as the optimal amplification degree.

In addition, the amplification magnitude determination module (135) of the present invention may be formed to acquire, in the case where optimization is required as shown in FIG. 10C, an SNR by starting scanning from P1 and increasing the amplification degree to Pn, and set the maximum SNR point as the optimal amplification degree.

In addition, the amplification magnitude determination module (135) of the present invention may be formed to set, in the case where rapid setting to a desired SNR level is required as shown in FIG. 10D, a point, where a value within a preset error range from the desired SNR is obtained by performing a zigzag scan starting from Pn, as the optimal amplification degree.

Although FIGS. 10A to 10D illustrate only a specific situation, the present invention is not limited thereto, and may be formed to determine an amplification magnitude by setting a specific SNR point as the optimal amplification degree according to various settings and conditions.

The optimal amplification degree described above may be determined using the analog-to-digital converter of the signal conversion module (131). The signal passing through the analog-to-digital converter is output as a signal of 0 and 1. At this point, the analog-to-digital converter has a characteristic of outputting a digital signal of a preset magnitude when a signal larger than the preset magnitude is acquired. That is, in the present invention, it may be formed such that in the case where the same digital signal is output, when the SNR of Pn is measured by increasing the amplification degree as shown in FIGS. 10A and 10C, the immediately previous amplification degree is set as the optimal amplification degree, using the characteristic of the analog-digital converter. In addition, contrary to FIGS. 10A and 10C, it may be formed such that in the case where a change begins to appear while the same digital signal is output, when the SNR of Pn is measured by gradually decreasing the amplification degree as shown in FIG. 10B, the immediately previous amplification degree may be set as the optimal amplification degree.

Here, the amplification magnitude determination module (135) according to an embodiment of the present invention determines whether or not to change the amplification degree using the determination time for changing the amplification degree, and when it is determined that the amplification degree needs to be changed, it may change the amplification degree.

The amplification magnitude determination module (135) determines whether or not to change the amplification degrees of the first biological activity signal and the second biological activity signal acquired using a determination time preset by the manager. When an SNR of the biological activity signal is stably acquired, the amplification magnitude determination module (135) does not need to change the amplification degree. However, when the SNR is not acquired as a desired SNR, the amplification magnitude determination module (135) needs to change the amplification degree. As a more detailed example, when the magnitude of a biological activity changes due to the movement of a user, the magnitudes of the acquired first biological activity signal and second biological activity signal are changed, and therefore, when the first biological activity signal and the second biological activity signal are amplified using the same amplification degree, an SNR different from the desired SNR may be acquired.

Accordingly, the amplification magnitude determination module (135) may determine whether or not to change the amplification degree through a buffer (determination reference time) for determining whether or not to change the amplification degree before determining the magnitude of amplification using FIGS. 10A to 10D described above.

Generally, an analog-to-digital converter has a maximum value and a minimum value that can be output. When the magnitude of the initially acquired biological activity signal decreases or increases, the value of the digital signal output from the analog-to-digital converter also decreases or increases. At this point, when the magnitude of the biological activity signal goes out of the output range of the analog-to-digital converter, the digital signal output after passing through the analog-to-digital converter has only the minimum or maximum value although the biological activity signal changes, and this may be a situation in which a high-resolution biological activity signal desired to derive through the present invention cannot be acquired.

Accordingly, in an embodiment of the present invention, the amplification magnitude determination module (135) may be formed to acquire the digital signal value of the biological activity signal, determine that the current amplification degree is over-amplification or under-amplification when the acquired digital signal value is continuously output as the minimum value or maximum value during a buffer time, which is a preset determination reference time, and change the amplification degree by determining the amplification degree as shown in FIG. 10A or 10C using the determination result.

In another embodiment of the present invention, a situation of the amplification magnitude determination module (135), in which rapid setting of a desired SNR is required as shown in FIG. 10D, may be, for example, the initial setting step of the system (1) of the present invention. In addition, a situation of the amplification magnitude determination module (135), in which optimization of desired SNR setting as shown in FIG. 10B is required, may be a case where the acquired biological activity signal has a digital signal value that does not deviate from a preset amplification degree reset range during a preset optimization determination time.

Meanwhile, the signal extraction unit (15) according to an embodiment of the present invention may be formed to output a digital-processed biological activity signal to extract an amplified signal for each preset biological activity signal category. To this end, the signal extraction unit (15) of the present invention may include a digital-processed biological activity signal output determination module (151) and a digital-processed biological activity signal output module (153), as shown in FIG. 4.

The digital-processed biological activity signal output determination module (151) may determine to output the digital-processed biological activity signal when the signal-to-noise ratio is higher than a preset magnitude, and not to output the digital-processed biological activity signal when the signal-to-noise ratio is lower than the preset magnitude. Here, when it is determined not to output the digital-processed biological activity signal, the digital-processed biological activity signal may be transmitted to the amplification magnitude determination module (135) described above and used to determine the amplification magnitude of the digital-processed biological activity signal.

The digital-processed biological activity signal output module (153) is formed to determines, when it is determined to output the digital-processed biological activity signal, a preset biological activity signal category to which the digital-processed biological activity signal corresponds, and separately output the digital-processed biological activity signal in each signal category. The digital-processed biological activity signal output module (135) is formed to categorize the digital-processed biological activity signal according to preset biological activity signal categories, and here, the preset biological activity signal categories may be, for example, a biological signal, an activity signal, and the like. The digital-processed biological activity signal output module (153) may be formed to output the corresponding digital-processed biological activity signal through a port tagged with a corresponding category when the categorization is completed.

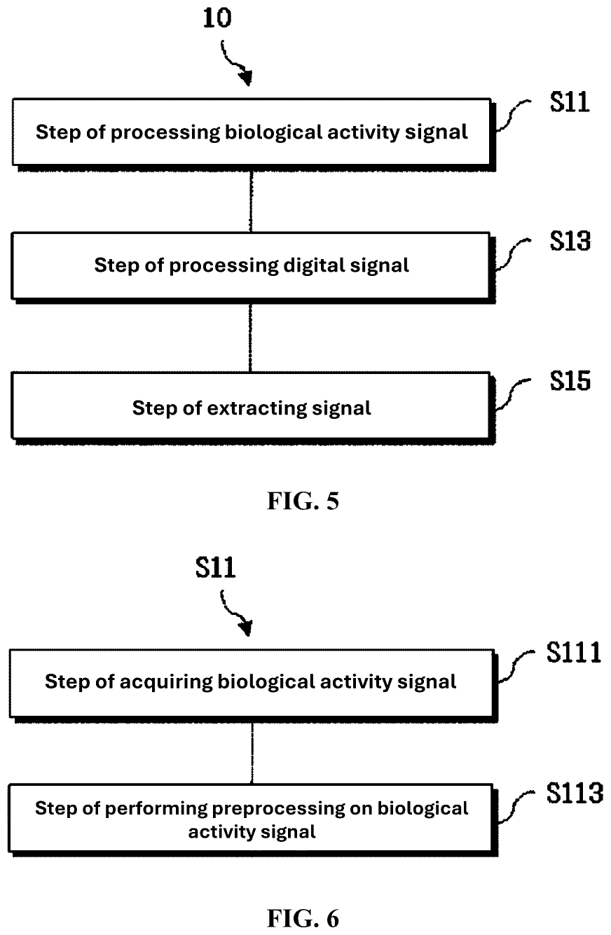

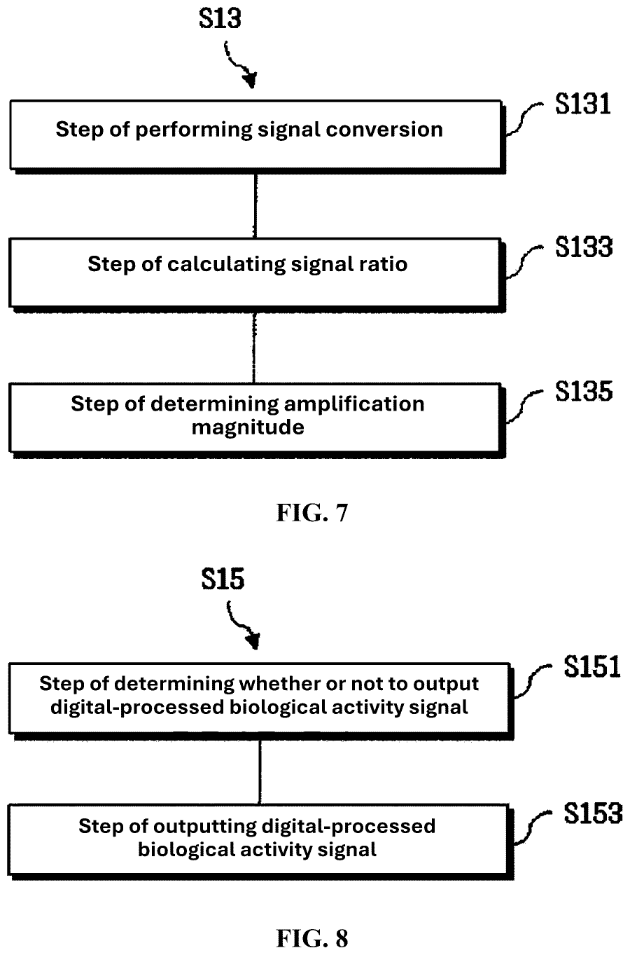

Meanwhile, FIGS. 5 to 8 show a high-resolution amplified biological activity signal acquisition method according to an embodiment of the present invention. FIG. 5 is a flowchart illustrating a high-resolution amplified biological activity signal acquisition method according to an embodiment of the present invention, FIG. 6 is a flowchart illustrating step S11 of FIG. 5, FIG. 7 is a flowchart illustrating step S13 of FIG. 5, and FIG. 8 is a flowchart illustrating step S15 of FIG. 5. Hereinafter, a high-resolution amplified biological activity signal acquisition method according to an embodiment of the present invention will be described in detail with reference to FIGS. 5 to 8, and the system of FIG. 1 will be used for convenience of explanation. However, the present invention is not necessarily limited to this, and may also be applied to devices or systems capable of performing similar operations.

A high-resolution amplified biological activity signal acquisition method (10) according to an embodiment of the present invention may be formed to acquire biological activity signals of a user using a radar device, classify the acquired biological activity signals into preset categories, and amplify and output the signals in each category. To this end, the high-resolution amplified biological activity signal acquisition method (10) according to an embodiment of the present invention is formed to include a step of processing a biological activity signal (S11), a step of processing a digital signal (S13), and a step of extracting a signal (S15), as shown in FIG. 5.

The step of processing a biological activity signal (S11) according to an embodiment of the present invention is formed to generate a preprocessed biological signal by acquiring a biological activity signal of a user using a radar device and performing preprocessing on the biological activity signal, by the biological activity signal processing unit. To this end, the step of processing a biological activity signal (S11) may be formed to include a step of acquiring a biological activity signal (S111) and a step of performing preprocessing on the biological activity signal (S113), as shown in FIG. 6.

The step of acquiring a biological activity signal (S111) is formed to acquire a biological activity signal of a user, which is information measured by a radar. The biological activity signal is formed to include a biological signal and an activity signal of the user. Here, the biological signal may be a signal measured for breathing, heart rate, and the like, and the activity signal may be a signal measured for movement of the user. When a biological activity signal is acquired, the step of acquiring a biological activity signal (S111) may acquire the biological signal included in the biological activity signal as a first biological activity signal, and acquire the activity signal included in the biological activity signal as a second biological activity signal.

The step of performing preprocessing on the biological activity signal (S113) is formed to generate a preprocessed biological activity signal by performing preprocessing on the biological activity signal. Here, the preprocessing may be performed on each of the first biological activity signal and the second biological activity signal. The preprocessing performed at the step of performing preprocessing on the biological activity signal (S113) may be a process of performing amplification on each of the first biological activity signal and the second biological activity signal using an amplifier at a preset magnitude, and applying a low pass filter of a preset band. Here, although the preset magnitude may be an amplification magnitude determined at the step of processing a digital signal (S13), which will be described below, an initially determined amplification magnitude may be used as an exception for the first biological activity signal and the second biological activity signal acquired initially.

The step of performing preprocessing on the biological activity signal (S113) in another embodiment of the present invention may use a variable amplifier to amplify the first biological activity signal and the second biological activity signal. The variable amplifier of the present invention may further include a band pass function that acquires the first biological activity signal and the second biological activity signal and passes only the frequencies of a preset band, and perform a function of amplifying the frequency of a corresponding band at a preset magnitude. In addition, in this embodiment, the low-pass filter of a preset band described above may be formed, when the biological activity signal passing through the variable amplifier is included in a frequency band other than the frequency band intended to be used in the present invention, to exclude the frequency band.

However, the present invention is not necessarily limited thereto, and when the magnitude is preset at the step of performing preprocessing on the biological activity signal (S113), the determined amplification magnitude may be used at the step of processing a digital signal (S13) for initial amplification.

The step of processing a digital signal (S13) is formed to generate a digital-processed biological signal by performing digital-processing on the preprocessed biological activity signal, by the digital signal processing unit. To this end, the step of processing a digital signal (S13) is formed to include a step of performing signal conversion (S131), a step of calculating a signal ratio (S133), and a step of determining an amplification magnitude (S135), as shown in FIG. 7.

The step of performing signal conversion (S131) is formed to digitalize the preprocessed biological activity signal. Here, the step of performing signal conversion (S131) may be, for example, an analog-to-digital converter (ADC). In addition, the step of performing signal conversion (S131) may be formed to further include a band pass filter to use a digital biological activity signal, which is the digitalized preprocessed biological activity signal converted through the analog-to-digital converter, only for a specific frequency band. Here, the specific frequency band of the band pass filter used at the step of performing signal conversion (S131) may be determined as a different frequency band according to the first biological activity signal and the second biological activity signal.

The digital biological activity signal, which is the preprocessed biological activity signal digitalized by passing through the step of performing signal conversion (S131), is used at the step of calculating a signal ratio (S133) to confirm an amplification degree and calculate a signal-to-noise ratio. The step of calculating a signal ratio (S133) may be formed to confirm the amplification degree of the digital biological activity signal and calculate the signal-to-noise ratio by performing conversion on the digital biological activity signal.

To this end, when a digital biological activity signal is acquired at the step of performing signal conversion (S131), first, the step of calculating a signal ratio (S133) may acquire information on the amplification degree by confirming the amplification degree of the digital biological activity signal, and calculate a signal-to-noise ratio by performing conversion on the digital biological activity signal. The conversion used herein may be, for example, fast Fourier transform (FFT). The digital biological activity signal, to which the fast Fourier transform (FFT) is applied, is converted into a pure biological activity signal, and the step of calculating a signal ratio (S133) may calculate the signal-to-noise ratio by comparing the digital biological activity signal and the pure biological activity signal.

The signal-to-noise ratio may be used at the step of outputting a signal (S15), which will be described below, to determine whether or not to output a corresponding digital biological activity signal according to the ratio value, and will be described below in further detail.

Meanwhile, the step of determining an amplification magnitude (S135) may be formed to determine, when the signal-to-noise ratio is acquired, an amplification magnitude by applying the amplification degree and the signal-to-noise ratio to a preset algorithm. Here, the amplification magnitude may be an amplification magnitude set at the step of performing preprocessing on the biological activity signal (S113) as described above. The step of determining an amplification magnitude (S135) may output the amplification magnitude as a PWM signal, and may be formed to control the amplification magnitude by outputting a voltage that may control the amplification degree through an integrator filter and providing the voltage to the amplifier of the step of performing preprocessing on the biological activity signal (S113).

FIGS. 10A-10D are exemplary diagrams showing a method of setting of an amplification magnitude in an amplification magnitude determination module or an amplification magnitude determination step of the present invention. Referring to FIGS. 10A-10D, the step of determining an amplification magnitude (S135) according to an embodiment of the present invention is configured to operate differently according to (a) a case of under-amplification, (b) a case of over-amplification (c) a case where optimization is required, and (d) a case where rapid setting to an SNR level is required.

First, in the case of under-amplification as shown in FIG. 10A, the step of determining an amplification magnitude (S135) may acquire an SNR (signal-to-noise ratio) by starting scanning from P1 and increasing the amplification degree to Pn, and determine an amplification magnitude by determining the maximum SNR point as the optimal amplification degree.

In addition, in the case of over-amplification as shown in FIG. 10B, contrary to FIG. 10A, the step of determining an amplification magnitude (S135) may acquire an SNR by starting scanning from P1 and decreasing the amplification degree to Pn, and set the maximum SNR point as the optimal amplification degree.

In addition, the step of determining an amplification magnitude (S135) of the present invention may be formed to acquire, in the case where optimization is required as shown in FIG. 10C, an SNR by starting scanning from P1 and increasing the amplification degree to Pn, and set the maximum SNR point as the optimal amplification degree.

In addition, the step of determining an amplification magnitude (S135) of the present invention may be formed to set, in the case where rapid setting to a desired SNR level is required as shown in FIG. 10D, a point, where a value within a preset error range from the desired SNR is obtained by performing a zigzag scan starting from Pn, as the optimal amplification degree.

Although FIGS. 10A to 10D illustrate only a specific situation, the present invention is not limited thereto, and may be formed to determine an amplification magnitude by setting a specific SNR point as the optimal amplification degree according to various settings and conditions.

The optimal amplification degree described above may be determined using the analog-to-digital converter of the step of performing signal conversion (S131). The signal passing through the analog-to-digital converter is output as a signal of 0 and 1. At this point, the analog-to-digital converter has a characteristic of outputting a digital signal of a preset magnitude when a signal larger than the preset magnitude is acquired. That is, in the present invention, it may be formed such that in the case where the same digital signal is output, when the SNR of Pn is measured by increasing the amplification degree as shown in FIGS. 10A and 10C, the immediately previous amplification degree is set as the optimal amplification degree, using the characteristic of the analog-digital converter. In addition, contrary to FIGS. 10A and 10C, it may be formed such that in the case where a change begins to appear while the same digital signal is output, when the SNR of Pn is measured by gradually decreasing the amplification degree as shown in FIG. 10B, the immediately previous amplification degree may be set as the optimal amplification degree.

Here, the step of determining an amplification magnitude (S135) according to an embodiment of the present invention determines whether or not to change the amplification degree using the determination time for changing the amplification degree, and when it is determined that the amplification degree needs to be changed, it may change the amplification degree.

The step of determining an amplification magnitude (S135) determines whether or not to change the amplification degrees of the first biological activity signal and the second biological activity signal acquired using a determination time preset by the manager. When an SNR of the biological activity signal is stably acquired, the step of determining an amplification magnitude (S135) does not need to change the amplification degree. However, when the SNR is not acquired as a desired SNR, the step of determining an amplification magnitude (S135) needs to change the amplification degree. As a more detailed example, when the magnitude of a biological activity changes due to the movement of a user, the magnitudes of the acquired first biological activity signal and second biological activity signal are changed, and therefore, when the first biological activity signal and the second biological activity signal are amplified using the same amplification degree, an SNR different from the desired SNR may be acquired.

Accordingly, the step of determining an amplification magnitude (S135) may determine whether or not to change the amplification degree through a buffer (determination reference time) for determining whether or not to change the amplification degree before determining the magnitude of amplification using FIGS. 10A to 10D described above.

Generally, an analog-to-digital converter has a maximum value and a minimum value that can be output. When the magnitude of the initially acquired biological activity signal decreases or increases, the value of the digital signal output from the analog-to-digital converter also decreases or increases. At this point, when the magnitude of the biological activity signal goes out of the output range of the analog-to-digital converter, the digital signal output after passing through the analog-to-digital converter has only the minimum or maximum value although the biological activity signal changes, and this may be a situation in which a high-resolution biological activity signal desired to derive through the present invention cannot be acquired.

Accordingly, in an embodiment of the present invention, the step of determining an amplification magnitude (S135) may be formed to acquire the digital signal value of the biological activity signal, determine that the current amplification degree is over-amplification or under-amplification when the acquired digital signal value is continuously output as the minimum value or maximum value during a buffer time, which is a preset determination reference time, and change the amplification degree by determining the amplification degree as shown in FIG. 10A or 10C using the determination result.

In another embodiment of the present invention, a situation at the step of determining an amplification magnitude (S135), in which rapid setting of a desired SNR is required as shown in FIG. 10D, may be, for example, the initial setting step of the method (10) of the present invention. In addition, a situation at the step of determining an amplification magnitude (S135), in which optimization of desired SNR setting as shown in FIG. 10B is required, may be a case where the acquired biological activity signal has a digital signal value that does not deviate from a preset amplification degree reset range during a preset optimization determination time.

Meanwhile, the step of extracting a signal (S15) according to an embodiment of the present invention may be formed to output, by the signal extraction unit, a digital-processed biological activity signal to extract an amplified signal for each preset biological activity signal category. To this end, the step of extracting a signal (S15) of the present invention may include a step of determining whether or not to output a digital-processed biological activity signal (S151) and a step of outputting a digital-processed biological activity signal (S153), as shown in FIG. 8.

The step of determining whether or not to output a digital-processed biological activity signal (S151) may determine to output the digital-processed biological activity signal when the signal-to-noise ratio is higher than a preset magnitude, and not to output the digital-processed biological activity signal when the signal-to-noise ratio is lower than the preset magnitude. Here, when it is determined not to output the digital-processed biological activity signal, the digital-processed biological activity signal may be transmitted to the step of determining an amplification magnitude (S135) described above and used to determine the amplification magnitude of the digital-processed biological activity signal.

The step of outputting a digital-processed biological activity signal (S153) is formed to determines, when it is determined to output the digital-processed biological activity signal, a preset biological activity signal category to which the digital-processed biological activity signal corresponds, and separately output the digital-processed biological activity signal in each signal category. The digital-processed biological activity signal output module (135) is formed to categorize the digital-processed biological activity signal according to preset biological activity signal categories, and here, the preset biological activity signal categories may be, for example, a biological signal, an activity signal, and the like. The step of outputting a digital-processed biological activity signal (S153) may be formed to output the corresponding digital-processed biological activity signal through a port tagged with a corresponding category when the categorization is completed.

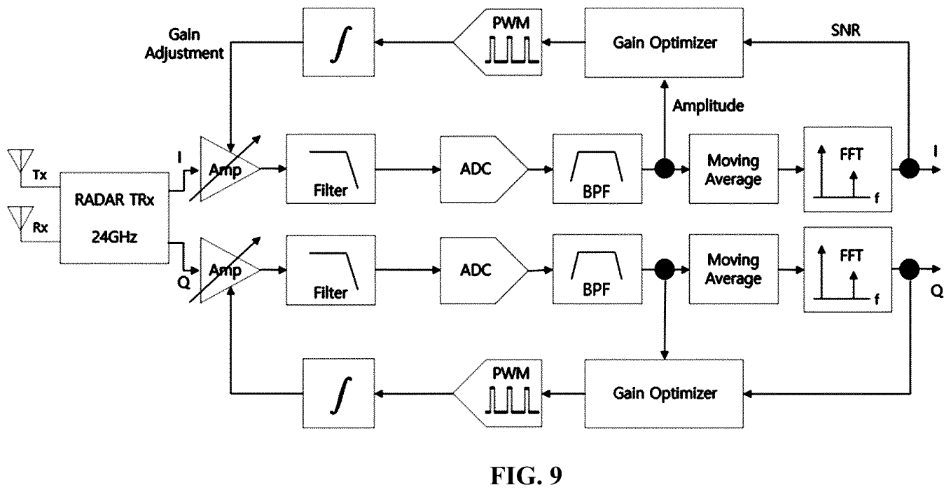

Meanwhile, FIG. 9 is a conceptual view showing the present invention of FIGS. 1 and 5. FIG. 9 shows the flow (concept) of a biological activity signal acquired through a radar using the system and method of the present invention until it is amplified and output. Referring to FIG. 9, the system and method of the present invention first acquires a biological activity signal including a biological signal and an activity signal using a radar. The acquired biological signal and activity signal are expressed as I and Q respectively for convenience of distinction in FIG. 9. The biological signal (first biological activity signal) and the activity signal (second biological activity signal) are each amplified through an amplifier. Here, the amplified magnitude may be determined using a preset amplification value or may be determined using the signal-to-noise ratio of a corresponding biological activity signal.

The amplified biological activity signal is formed to pass through a filter and delete a predetermined frequency band. Here, the filter may be a low pass filter. The biological activity signal passing through the low-pass filter, from which signals in a band lower than a preset frequency band are deleted, passes through an analog-to-digital converter to be converted into a digital signal, and passes through a band-pass filter to be transformed to leave only a specific frequency band. After measuring the amplification degree of the digital biological activity signal of only the remaining specific frequency band, the present invention performs FFT on the digital biological activity signal and acquires a signal-to-noise ratio using the result of the FFT. The present invention may be formed to acquire, when the signal-to-noise ratio and the amplification degree are acquired, an optimal amplification degree using these two types of information, and control the amplification degree by outputting the acquired amplification degree through a PWM signal and controlling the voltage of the amplifier.

Meanwhile, the present invention is formed to output, when the signal-to-noise ratio is higher than a preset magnitude, a corresponding digital biological activity signal without changing the amplification degree, and when the digital biological activity signal is output, the digital biological activity signal may be output through a different port according to the category of the signal.

Although an embodiment of the present invention has been described above, the spirit of the present invention is not limited to the embodiment presented in the present specification, and although those skilled in the art who understand the spirit of the present invention may easily suggest other embodiments by adding, changing, deleting, or supplementing components within the scope of the same spirit, it will be said that this is also within the scope of the present invention.

1. A high-resolution amplified biological activity signal acquisition system comprising:
a biological activity signal processing unit for generating a preprocessed biological activity signal by acquiring a biological activity signal of a user using a radar device and performing preprocessing on the biological activity signal;
a digital signal processing unit for generating a digital-processed biological activity signal by performing digital-processing on the preprocessed biological activity signal; and
a signal extraction unit for outputting the digital-processed biological activity signal to extract an amplified signal for each preset biological activity signal category.

2. The system according to claim 1, wherein the biological activity signal processing unit includes:
a biological activity signal acquisition module for acquiring the biological activity signal including a first biological activity signal that is a biological signal and a second biological activity signal that is an activity signal; and
a biological activity signal preprocessing module for generating the preprocessed biological activity signal by performing preprocessing on the biological activity signal.

3. The system according to claim 2, wherein the preprocessing is a process of performing amplification at a preset magnitude using an amplifier on each of the first biological activity signal and the second biological activity signal, and applying a low pass filter of a preset band.

4. The system according to claim 3, wherein the digital signal processing unit includes:
a signal conversion module for digitalizing the preprocessed biological activity signal;
a signal ratio calculation module for confirming an amplification degree of a digital biological activity signal, which is the digitalized preprocessed biological activity signal, and calculating a signal-to-noise ratio by performing conversion on the digital biological activity signal; and
an amplification magnitude determination module for determining the preset magnitude by applying the amplification degree and the signal-to-noise ratio to a preset algorithm.

5. The system according to claim 4, wherein the signal extraction unit includes:
a digital-processed biological activity signal output determination module for determining to output the digital biological activity signal when the signal-to-noise ratio is higher than a preset magnitude, and not to output the digital biological activity signal when the signal-to-noise ratio is lower than the preset magnitude; and
a digital-processed biological activity signal output module for determining, when it is determined to output the digital-processed biological activity signal, the preset biological activity signal category to which the digital-processed biological activity signal corresponds, and separately outputting the digital-processed biological activity signal in each signal category.

6. A high-resolution amplified biological activity signal acquisition method comprising:
a biological activity signal processing step of generating a preprocessed biological activity signal by acquiring a biological activity signal of a user using a radar device and performing preprocessing on the biological activity signal, by a biological activity signal processing unit;
a digital signal processing step of generating a digital-processed biological activity signal by performing digital-processing on the preprocessed biological activity signal, by a digital signal processing unit; and
a signal extraction step of outputting the digital-processed biological activity signal to extract an amplified signal for each preset biological activity signal category, by a signal extraction unit.

7. The method according to claim 6, wherein the biological activity signal processing step includes:
a step of acquiring the biological activity signal including a first biological activity signal that is a biological signal and a second biological activity signal that is an activity signal; and
a biological activity signal preprocessing step of generating the preprocessed biological activity signal by performing preprocessing on the biological activity signal.

8. The method according to claim 7, wherein the preprocessing is a process of performing amplification at a preset magnitude using an amplifier on each of the first biological activity signal and the second biological activity signal, and applying a low pass filter of a preset band.

9. The method according to claim 8, wherein the digital signal processing step includes:
a step of digitalizing the preprocessed biological activity signal;
a signal ratio calculation step of confirming an amplification degree of a digital biological activity signal, which is the digitalized preprocessed biological activity signal, and calculating a signal-to-noise ratio by performing conversion on the digital biological activity signal; and
an amplification magnitude determination step of determining the preset magnitude by applying the amplification degree and the signal-to-noise ratio to a preset algorithm.

10. The method according to claim 9, wherein the signal extraction step includes:
a digital-processed biological activity signal output determination step of determining to output the digital biological activity signal when the signal-to-noise ratio is higher than a preset magnitude, and not to output a digital biological activity signal when the signal-to-noise ratio is lower than the preset magnitude; and
a digital-processed biological activity signal output step of determining, when it is determined to output the digital-processed biological activity signal, the preset biological activity signal category to which the digital-processed biological activity signal corresponds, and separately outputting the digital-processed biological activity signals in each signal category.
